(19) United States
(12) Patent Application Publication
(10) Pub. No.: US 2020/0227168 A1
(43) Pub. Date: Jul. 16, 2020

(54) MACHINE LEARNING IN FUNCTIONAL CANCER ASSAYS

(71) Applicant: Travera LLC, Cambridge, MA (US)

(72) Inventors: Rob Kimmerling, Cambridge, MA (US); Selim Olcum, Cambridge, MA (US); Clifford Reid, Pacifica, CA (US); Mark Stevens, Cambridge, MA (US)

(21) Appl. No.: 16/739,814

(22) Filed: Jan. 10, 2020

Related U.S. Application Data
(60) Provisional application No. 62/790,804, filed on Jan. 10, 2019.

(51) Int. Cl. G16H 50/20; G06N 3/08; G06N 20/20; G06N 5/00; G16H 15/00; G16H 70/60

(52) U.S. Cl. CPC G16H 50/20; G06N 3/088; G16H 70/60; G06N 5/003; G16H 15/00; G06N 20/20

(57) ABSTRACT

The invention provides methods that use machine learning to discover clinical data patterns that are predictive of disease, such as cancer. Clinical data from across a population is provided as input to a machine learning system. The machine learning system discovers associations in data from a plurality of data sources obtained from a population and correlates the associations to cancer status of patients in the population. The methods may further include providing patient data from an individual and predicting, by the machine learning system, a cancer state (e.g., the presence of cancer and a determination of a stage or progression of the cancer, if present) for the individual when the patient data presents one or more of the discovered associations.

### CROSS-REFERENCE TO RELATED APPLICATIONS

This application claims the benefit of, and priority to, U.S. Provisional Patent Application No. 62/790,804, filed Jan. 10, 2019, the contents of which are incorporated by reference.

### TECHNICAL FIELD

The disclosure relates to methods for evaluating disease.

### BACKGROUND

Cancer is a global health issue that causes millions of deaths worldwide every year. Standard treatments typically are based on the evaluation of a cell lines, animal models, and human subjects. Still, individual patient response to a drug or therapy are often variable and unpredictable even for cancers of identical tissue origin and common histology. Consequently, while current treatments benefit some patients, other patients may receive little to no benefit and may further suffer from adverse reactions. Accordingly, while there are many different cancer treatments available, there is limited ability to effectively predict how an individual patient will respond to a particular treatment, which may lead to extended periods of time in which a patient endures a treatment that simply isn't working as intended.

### SUMMARY

The invention provides systems and methods that use machine learning to discover clinical data patterns that are predictive of disease, such as cancer. Clinical data from across a population is provided as input to a machine learning system. The clinical data includes a training data set, which includes functional biomarker measurements from a plurality of patient samples, each having a known cancer status. The machine learning system discovers associations in the training data and correlates cancer statuses to functional biomarker measurement results. In particular, the machine learning system processes the training data set and discovers latent patterns that are predictive of cancer, including a stage or progression of the cancer, as well as treatments that are effective and ineffective. After repeatedly finding associations among data (i.e., biophysical measurements and/or genomic data) across the population, the machine learning system learns the association and its correlation to cancer status. The system is robust in that it can learn any arbitrary number of patterns or associations across population data in a manner that is free from a priori expectations that a health professional may have in mind. The machine learning system can discover associations over any span of time, without bias, and reliably build the correlations between those associations and cancer states.

Due to the ability of the machine learning system to discover associations among a training data set comprised of functional biomarker measurements that correlate to cancer statuses, the system is useful in predicting cancer status for individuals. For example, the machine learning system is able receive patient data from an individual and predict a cancer status for the individual when the patient data presents one or more of the discovered associations. In particular, the patient data may include functional biomarker measurements of a patient sample either known to be, or suspected of being, cancerous. The functional biomarker measurements from an individual are similar to the functional biomarker measurements used in the training data set, wherein such measurements include biophysical data (i.e., growth of live cells by measuring mass or change in mass in the cells) and genetic data of the cells.

Upon detecting that association among the patient data for the individual, the machine learning system further generates a report providing information related to the cancer evaluation, including, but not limited to, specific data associated with the patient sample having undergone testing, whether the test is positive for cancer, a determination of a stage or progression of cancer, and a customized treatment plan tailored to an individual patient's cancer diagnosis. The report may further provide predictive information, such as a prediction of risk of cancer for this patient in the future. As such, the report provided by systems and method of the present invention allows the health professional to initiate additional tests and begin treatment interventions far earlier than would otherwise have been possible.

Instruments of the disclosure are used to measure cellular functions that embody the viability of the cells. The instruments may be used to measure the growth of the cells by measuring mass or change in mass in the cells. In a tissue sample containing only non-cancerous differentiated somatic cells, the cells will tend to exhibit stable masses whereas cancer cells may exhibit growth as the accumulation of mass. Similarly, known cancer cells that are responding favorably to therapeutic may exhibit loss of mass. Instruments of the disclosure can make sensitive and precise measurements of mass or change in mass through the use of a suspended microchannel resonator. The instruments use a structure such as a cantilever that contains a fluidic microchannel. Living cells are flowed through the structure, which is resonated and its frequency of resonation is measured. The frequency at which a structure resonates is dependent on its mass and by measuring the frequency of at which the cantilever resonates, the instrument can compute a mass, or change in mass, of a living cell in the fluidic microchannel. By flowing the isolated living cells from the tissue sample through such devices, one may observe the functions of those cells, such as whether they are growing and accumulating mass or not. The mass accumulation or rate of mass accumulation can be related to clinically important property such as the presence of cancer cells or the efficacy of a therapeutic on cancer cells.

Thus, the functional biomarker measurements of the training data set may include measurements of functional properties of living cells in a tissue sample or bodily fluid sample. Those functional properties provide a valuable marker of cancer activity. Once the measurements are made, those living cells are available for further study, such as genome sequencing or other measurements. As such, in some embodiments, the training data set further includes at least one other source of data associated with known cancer statuses, such as, for example, genomic data. Accordingly, a training data set may include both biophysical data and genetic data to thereby provide a detailed characterization of a given cell, in turn allowing for a more comprehensive cancer evaluation.

Aspects of the invention are accomplished by providing, to a computing system, a training data set comprising functional biomarker measurements from a plurality of patient samples each having a known cancer status, and associating the functional biomarker measurements with the cancer statuses. The method further includes obtaining a sample from a patient suspected of having cancer, measuring a functional biomarker of one or more live cells isolated from the sample, and inputting data obtained in the measuring step into the computing system. The method further includes correlating, via the computing system, the data with the cancer statuses and reporting results of the correlating step to the patient.

In some embodiments, the measuring step includes obtaining measurements from one or more assays performed on the sample from a patient. For example, live cells may be obtained from a sample (tissue of bodily fluid) of a patient. The sample may include a fine needle aspirate, a biopsy, or a bodily fluid from a patient suspected of having cancer. Upon being isolated from the sample, the live cells undergo a first assay to obtain a functional property of the live cells, specifically a functional biomarker measurement. In particular, the first assay involves loading individual live cells into a functional biomarker measurement instrument, such as, for example, a suspended microchannel resonator (SMR) measurement instrument and flowing the live cells through the SMR. The SMR may be used to precisely measure biophysical properties, such as mass and mass changes, of a single cell flowing therethrough. The mass change may be mass accumulation rate (MAR). The live cells remaining in a living state upon passing through the SMR instrument, such that they are accessible for one or more additional live cell assays downstream from the first assay. Accordingly, the live cells may undergo at least a second assay to obtain additional measurements. As such, the measuring step may further include performing at least a second assay on the live cells to obtain additional data, which may include genome sequencing to obtain sequence data.

As such, the inputting step includes inputting the data obtained from the first assay (i.e., single-cell functional biomarker measurements, such as mass accumulation rate) and data obtained from the one or more additional assays (e.g., single-cell genetic data). The computing system is then able to correlate such data with the cancer statuses obtained via the training data set to detect any association, and further provide a report of the evaluation results based on the correlation step. In particular, the report provides information related to the cancer evaluation, including, but not limited to, whether the sample tested positive for cancer, a determination of a stage or progression of cancer, and a customized treatment plan tailored to an individual patient's cancer diagnosis. As such, the methods of the present invention can improve outcomes of cancer treatment, avoid any unnecessary cancer treatment, and reduce overall healthcare costs.

In some embodiments, the computing system comprises a machine learning system selected from the group consisting of a random forest, a support vector machine, a Bayesian classifier, and a neural network.

In some embodiments, the computing system comprises an autonomous machine learning system that associates the functional biomarker measurements with the known cancer statuses in an unsupervised manner. The autonomous machine learning system may include a deep learning neural network that includes an input layer, a plurality of hidden layers, and an output layer. The autonomous machine learning system may represent the training data set using a plurality of features, wherein each feature comprises a feature vector. In some embodiments, the autonomous machine learning system may comprise a random forest.

In some embodiments, the method further comprises operating a machine learning system to learn relationships among cancer statuses, treatment options, depth of response, known treatment efficacies, and progression free survival. The method may further include selecting, by the machine learning system, one or more recommended treatments for the patient based, at least in part, on the results of the correlating step and learned relationships. In some embodiments, one or more of the training data set, cancer statuses, treatment options, depth of response, known treatment efficacies, and progression free survival may be obtained from one or more publicly available data repositories.

### BRIEF DESCRIPTION OF THE DRAWINGS

FIG. 1 diagrams a method for disease evaluation.

FIG. 2 shows a machine learning system according to certain embodiments.

FIG. 3 diagrams a system for predicting cancer status by methods of the invention.

FIG. 4 shows a report as may be provided.

FIG. 5 shows measurement of biophysical properties of a single cell.

FIG. 6 shows a microchannel flow path of a SMR consistent with the present disclosure.

FIG. 7 shows a serial suspended microchannel resonator (sSMR) array.

FIG. 8 diagrams an SMR detection system consistent with the present disclosure.

FIG. 9 diagrams a sequencing workflow consistent with the present disclosure.

### DETAILED DESCRIPTION

The invention provides systems and methods that use machine learning to discover clinical data patterns that are predictive of disease, such as cancer. Clinical data from across a population is provided as input to a machine learning system. The clinical data includes a training data set comprised of functional biomarker measurements from a plurality of patient samples, each having a known cancer status. The functional biomarker measurements are indicative of how living cells function. When the cells are obtained from a person suspected of having cancer, the measurements can show that cancer cells are present and measurements over time can show the progress of the cancer or how the cancer is reacting to stimulus such as a therapeutic treatment. The measurements can be made from tissue biopsy samples or bodily fluid samples to measure functional properties of living tumor cells, for example.

The functional biomarker measurements of the training data set may include measurements of functional properties of living cells in a tissue sample or bodily fluid sample. Those functional properties provide a valuable marker of cancer activity. Instruments of the disclosure are used to measure cellular functions that embody the viability of the cells. In particular, such instruments may be used to measure the growth of the cells by measuring mass or change in mass in the cells. In a tissue sample or bodily fluid sample containing only non-cancerous differentiated somatic cells, the cells will tend to exhibit stable masses whereas cancer cells may exhibit growth as the accumulation of mass. Similarly, known cancer cells that are responding favorably to therapeutic may exhibit loss of mass. Instruments of the disclosure can make sensitive and precise measurements of mass or change in mass through the use of a suspended microchannel resonator. The instruments use a structure such as a cantilever that contains a fluidic microchannel. Living cells are flowed through the structure, which is resonated and its frequency of resonation is measured. The frequency at which a structure resonates is dependent on its mass and by measuring the frequency of at which the cantilever resonates, the instrument can compute a mass, or change in mass, of a living cell in the fluidic microchannel. By flowing the isolated living cells from the tissue sample through such devices, one may observe the functions of those cells, such as whether they are growing and accumulating mass or not. The mass accumulation or rate of mass accumulation can be related to clinically important property such as the presence of cancer cells or the efficacy of a therapeutic on cancer cells.

Once the measurements are made, those living cells are available for further study, such as genome sequencing or other measurements. As such, in some embodiments, the training data set further includes at least one other source of data associated with known cancer statuses, such as, for example, genomic data. Accordingly, a training data set may include both biophysical data and genetic data to thereby provide a detailed characterization of a given cell, in turn allowing for a more comprehensive cancer evaluation.

The machine learning system discovers associations in data from the plurality of data sources obtained from the population and correlates the associations to cancer statuses of patients in the population. In particular, the machine learning system processes the clinical data (i.e., the training data set) and discovers latent patterns that are predictive of the cancer, including a stage or progression of the cancer, as well as treatments that are effective and ineffective. After repeatedly finding that association between data entries (i.e., biophysical measurements and/or genomic data) across the population, the machine learning system learns the association and its correlation to the future diagnosis. The system is robust in that it can learn any arbitrary number of patterns or associations across the population data and it is free from a priori expectations that a health professional may have in mind. The machine learning system can discover associations over any span of time, without bias, and reliably build the correlations between those associations and future cancer states.

Due to the ability of the machine learning system to discover associations among a training data set comprised of functional biomarker measurements that correlate to cancer statuses, the system is useful in predicting cancer status for individuals. For example, the machine learning system is able receive patient data from an individual and predict a cancer state for the individual when the patient data presents one or more of the discovered associations. In particular, the patient data may include functional biomarker measurements of a patient sample either known to be, or suspected of being, cancerous. The functional biomarker measurements from an individual are similar to the functional biomarker measurements used in the training data set, wherein such measurements include biophysical data (i.e., growth of live cells by measuring mass or change in mass in the cells) and genetic data of the cells.

Upon detecting that association among the patient data for the individual, the machine learning system further generates a report providing information related to the cancer evaluation, including, but not limited to, specific data associated with the patient sample having undergone testing, whether the test is positive for cancer, a determination of a stage or progression of cancer, and a customized treatment plan tailored to an individual patient's cancer diagnosis. The report may further provide predictive information, such as a prediction of risk of cancer for this patient in the future. As such, the report provided by systems and method of the present invention allows the health professional to initiate additional tests and begin treatment interventions far earlier than would otherwise have been possible.

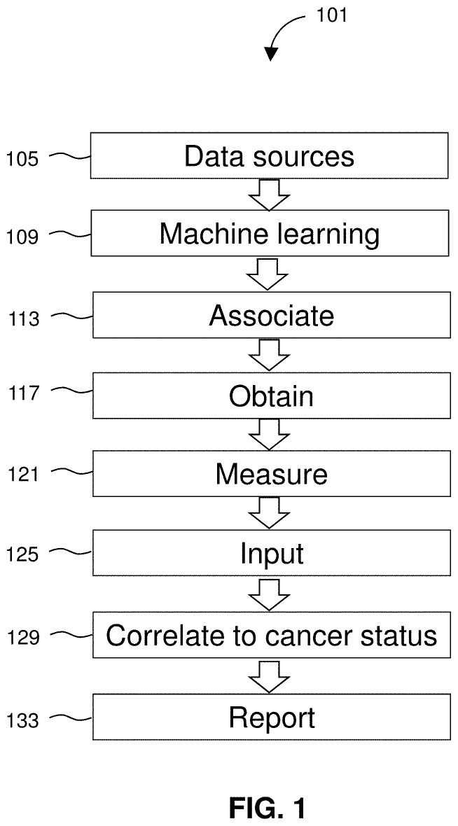

FIG. 1 diagrams a method 101 for evaluating a disease, specifically evaluating cancer. The method 101 includes accessing 105 multiple data sources of clinical data from a population. The clinical data may include a training data set including functional biomarker measurements from a plurality of patient samples, each having a known cancer status. The functional biomarker measurements may include, for example, biophysical properties of a single cancer cell or cancer-related immune cell of a patient sample. The biophysical properties may include mass or change in mass of a single cell (i.e., mass accumulation or rate of mass accumulation). In some embodiments, the training data set further includes at least one other source of data associated with known cancer statuses, such as, for example, genomic data. Accordingly, a training data set may include at least biophysical data and, in some instances, genetic data, of a single cell.

The method 101 further includes operating 109 a machine learning system. The machine learning system discovers associations in the clinical data from the population. In particular, the machine learning system associates 113 at least the functional biomarker measurements with the known cancer statuses, thereby establishing patterns that are predictive of the cancer, including a stage or progression of the cancer, as well as treatments that are effective and/or ineffective. In some embodiments, the machine learning system may learn relationships among cancer statuses, treatment options, depth of response, known treatment efficacies, and progression free survival. The training data set, cancer statuses, treatment options, depth of response, known treatment efficacies, and progression free survival may be obtained from one or more publicly available data repositories or data sources.

The method 101 further comprises obtaining 117 a sample from a patient suspected of having cancer. The sample may include, for example, a tissue sample (e.g., a fine needle aspirate or biopsy) or a bodily fluid sample from a patient suspected of having cancer. The method 101 further includes measuring 121 a functional biomarker of one or more live cells isolated from the sample. In some embodiments, the measuring step includes obtaining measurements from one or more assays performed on the sample from a patient to obtain one or more functional biomarker measurements of a patient sample either known to be, or suspected of being, cancerous. The functional biomarker measurements may generally be similar to the functional biomarker measurements used in the training data set, wherein such measurements include biophysical data (i.e., growth of live cells by measuring mass or change in mass in the cells) and genetic data of the cells.

For example, functional biomarker marker measurements may be obtained by performing at least a first assay on the one or more live cells to obtain single-cell biophysical properties, including, but not limited to, mass or change in mass of a single cell (i.e., mass accumulation or rate of mass accumulation). In some embodiments, as will be described in greater detail herein, the first assay may generally be performed with any functional biomarker measurement instrument, such as, for example, an instrument comprising a suspended microchannel resonator (SMR) or serial SMR (sSMR). The SMR may be used to precisely measure biophysical properties, such as mass and mass changes, of a single cell flowing therethrough. The mass change may be mass accumulation rate (MAR). When used with cancer cells, those changes provide a functional, universal biomarker by which medical professionals (e.g., oncologists) may monitor the progression of a cancer and determine how cancer cells respond to therapies.

Upon passing through the functional biomarker measurement instrument, the single cells remain viable and can be isolated downstream from the instrument where the cells may undergo subsequent use, such as testing in traditional assays. Accordingly, additional functional biomarker measurements may be obtained by performing at least a second assay on the live cells, either concurrently with the first assay, or downstream from the first assay, to obtain further data associated with the live cells, such as genomic data. As will be described in greater detail herein, the second assay may include genome sequencing, single cell transcriptomics, single cell proteomics, and single cell metabolomics. Yet still, in other embodiments, the second assay, or an additional assay, may include flow cytometry to analyze physical and/or chemical characteristics of the one or more cells, including the detection of biomarkers.

The method 101 further comprises inputting 125 data obtained in the measuring step into the computing system, wherein the machine learning system correlates 129 the data with the cancer statuses. The method 101 further includes providing 133 a report comprising results of the correlation step. In particular, the report may provide information related to the cancer evaluation, including, but not limited to, specific data associated with a sample having undergone testing, whether the test is positive for cancer, a determination of a stage or progression of cancer, and personalized treatment tailored to an individual patient's cancer. For example, in some embodiments, the machine learning system may be configured to select one or more recommended treatments for the patient based, at least in part, on the results of the correlating step and learned relationships. The report may further provide predictive information, such as a prediction of risk of cancer for this patient in the future. As such, the report provided by systems and method of the present invention allows the health professional to initiate additional tests and begin treatment interventions far earlier than would otherwise have been possible.

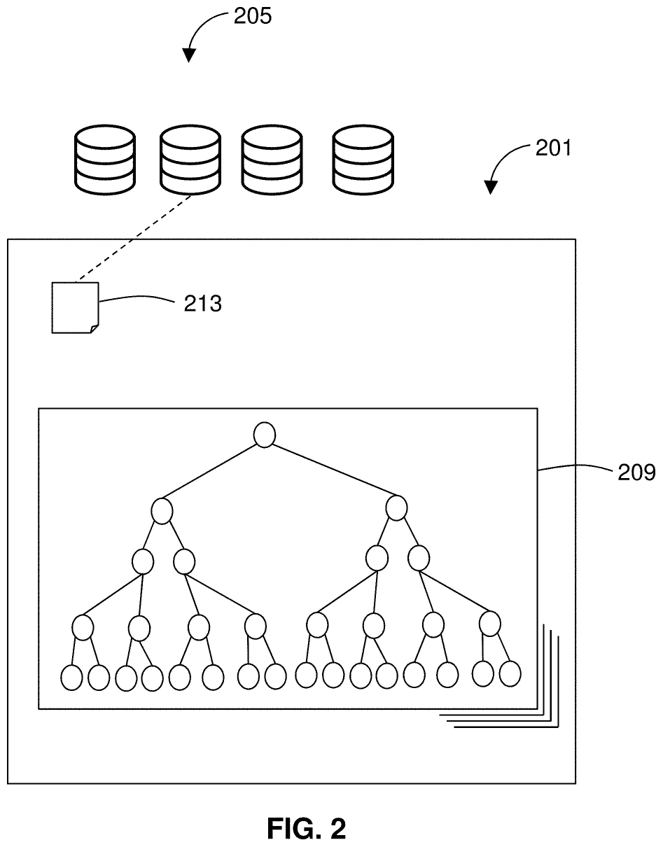

FIG. 2 shows a machine learning system 201 according to certain embodiments. The machine learning system 201 accesses data from a plurality of sources 205. Any suitable source of clinical data 205 may be provided 105 to the machine learning system 201. Generally, clinical data includes data that is collected during the course of ongoing patient care or as part of a formal clinical trial program. Types of clinical data include may include, but is not limited to, clinical trial data and test results, such as clinical laboratory assay results. For example, the clinical data includes a training data set comprised of functional biomarker measurements from a plurality of patient samples, each having a known cancer status.

The functional biomarker measurements may include, for example, biophysical properties of a single cancer cell or cancer-related immune cell of a patient sample. The biophysical properties may include mass or change in mass of a single cell (i.e., mass accumulation or rate of mass accumulation), which generally embody the viability of the cells. In some embodiments, the training data set further includes at least one other source of data associated with known cancer statuses, such as, for example, genomic data. The biophysical properties of a single cell, such as mass or growth rate, offer unique insights into a wide range of biological phenomena of a live cancer cell, including, but not limited to, basic patterns of single-cell mass and growth regulation, biophysical changes associated with immune cell activation, and cancer cell heterogeneity in the presence or absence of drug. Accordingly, a training data set including at least biophysical data, as well as molecular profiling, of a single cell allows for characterization of an underlying transcriptional program associated with cellular mass and growth rate variability in a range of normal and dysfunctional biological contexts.

In some embodiments, the clinical data may further include health/medical records, patient or disease registries, and/or health surveys. Disease registries are clinical information systems that track a narrow range of key data for certain chronic conditions, such as cancer. Registries often provide critical information for managing patient conditions. A disease registry may include, for example, the National Program of Cancer Registries. Health surveys generally include government or industry sponsored evaluations of population health. These surveys of the most common chronic conditions are generally conducted to provide prevalence estimates. National surveys are one of the few types of data collected specifically for research purposes, thus making it more widely accessible. Examples include the Medicare Current Beneficiary Survey, National Health & Nutrition Examination Survey (NHANES), The Medical Expenditure Panel Survey (MEPS), the National Center for Health Statistics, Center for Medicare & Medicaid Services Data Navigator, and the National Health and Aging Trends Study (NHATS). Clinical data may be obtained from clinical trials registries and databases such as ClinicalTrials.gov, WHO International Clinical Trials Registry Platform (ICTRP), the European Union Clinical Trials Database, the ISRCTN Registry (BioMed Central), or CenterWatch.

In preferred embodiments, the plurality of data sources 205 feed into the machine learning system 201. Any suitable machine learning system 201 may be used. For example, the machine learning system 201 may include one or more of a random forest, a support vector machine, a Bayesian classifier, and a neural network. In the depicted embodiment, the machine learning system 201 includes a random forest 209. In some embodiments, the computing system comprises an autonomous machine learning system that associates the functional biomarker measurements with the known cancer statuses in an unsupervised manner. The autonomous machine learning system may include a deep learning neural network that includes an input layer, a plurality of hidden layers, and an output layer. The autonomous machine learning system may represent the training data set using a plurality of features, wherein each feature comprises a feature vector.

The machine learning system 201 may access data from the plurality of sources 205 in any suitable format. However the initial format, the data ultimately can be understood to include a plurality of entries 213. Each entry preferably includes a datum, or a value, that provides information to the system 201. In some embodiments, each entry 213 in the data is specific to one patient from the population, and assigned to a pre-defined category. It will be understood that the data sources 205 may provide anonymized data. In such cases, each entry 213 is preferably specific to a patient and tracked to that patient by a patient ID value, which may be a random string or code. The external data sources 205 may provide the patient ID, or the machine learning system 201 may assign a patient ID to each entry 213. Each entry 213 preferably also has a category. For example, where a data entry 213 is a functional biomarker measurement, such as a mass accumulation rate (MAR), the category may be “MAR” (and the value for the entry 213 is a specific data point). In another example, where a data source 205 is an RNA-Seq assay for expression levels, a data entry 213 may be categorized as an expression level for one specific RNA and the value may be the expression level of that RNA. In yet one other example, where a data entry 213 is a patient's weight, the category may be “weight” and the value may be a mass in pounds or kilograms. The machine learning system 201 access one or more of the data sources 205 and discovers associations therein.

The machine learning system 201 discovers associations in data from the plurality of data sources obtained from the population and correlates the associations to cancer statuses of patients in the population. In particular, the machine learning system 201 processes the clinical data (i.e., the training data set) and discovers latent patterns that are predictive of the cancer, including a stage or progression of the cancer, as well as treatments that are effective and ineffective. After repeatedly finding that association between data entries (i.e., biophysical measurements and/or genomic data) across the population, the machine learning system 201 learns the association and its correlation to the future diagnosis. The system is robust in that it can learn any arbitrary number of patterns or associations across the population data and it is free from a priori expectations that a health professional may have in mind. The machine learning system 201 can discover associations over any span of time, without bias, and reliably build the correlations between those associations and future cancer states.

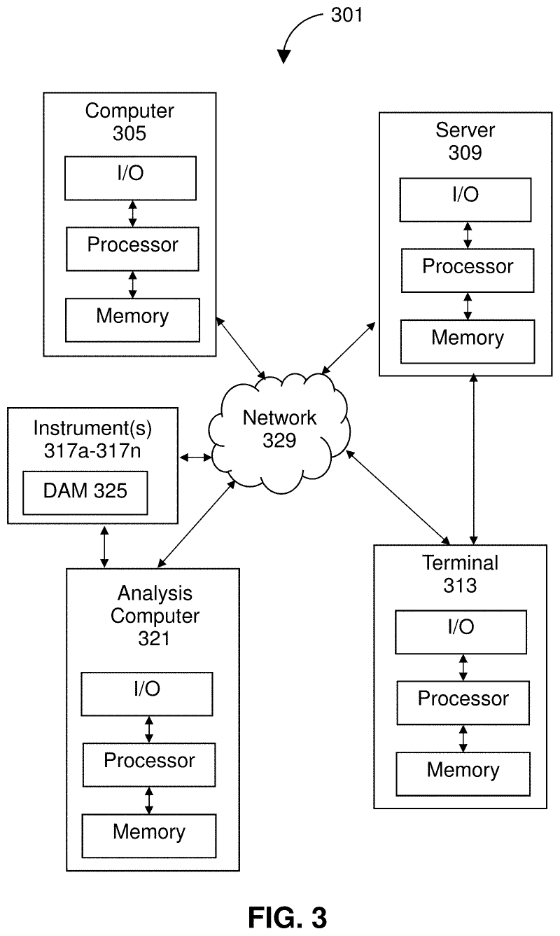

FIG. 3 diagrams a system 301 for predicting cancer status by methods of the invention. The system 301 includes at least one computer 305, such as a laptop or desktop computer, than can be accessed by a user to initiate methods of the invention and obtain results. The system 301 preferably also includes at least one server sub-system 309 and either or both of the computer 305 and the server sub-system 309 may include and provide the machine learning system 201. The server subsystem 309 may have a dedicated terminal computer 313 for accessing the server sub-system 309. Additionally, the system 301 operates in communication with a lab, such as a clinical services laboratory, which may include one or more analysis instruments 317a-317n. The one or more analysis instruments 317a-317n may be used to obtain one or more functional biomarker measurements (i.e., biophysical measurements and/or genomic data). For example, the one or more analysis instruments 317a-317n may include an instrument used to measure the growth of the cells by measuring mass or change in mass one or more living cells, an instrument used to obtain genomic data, such as a nucleic acid sequencing instrument, and any additional analysis instruments for performing additional assays on the one or more cells downstream.

Each analysis instrument 317a-317n may have its own data acquisition module 325, such as, for example, the flow cell and associated optical and electronic instruments of a nucleic acid sequencer, such as the sequencer sold under the trademark HISEQ or MISEQ by Illumina, Inc. The instrument 317a-317n may have its own built-in or connected instrument computer 321 as well. Any or all of the computer 305, server subsystem 309, terminal computer 313, instrument 317a-317n, and instrument computer 321 may exchange data over communications network 329, which may include elements of a local area network (LAN), a wide area network (WAN) the Internet, or combinations thereof. Each of computer 305, server subsystem 309, terminal computer 313, and instrument computer 321, when included, preferably includes at least one processor coupled to one or more input/output devices and a tangible, non-transitory memory subsystem. The I/O devices may include one or more of: monitor, keyboard, mouse, trackpad, touchpad, touchscreen, Wi-Fi card, cellular antenna, network interface cards, or others. The memory subsystem preferably includes one or more of RAM and a disc drive, such as a magnetic hard drive or solid state drive.

The system 301 contains instructions stored in the memory that are executable by one or more of processors to cause the system to discover, via the machine learning system 201, associations in data from a plurality of data sources 205 obtained from a population and correlate the associations to cancer status of patients in the population. In some embodiments, each entry 213 in the data is specific to one patient from the population, and assigned to a pre-defined category. The machine learning system 201 may receive a training data set comprising functional biomarker measurements from a plurality of patient samples, wherein each comprises a known cancer status. For the association step 109 of the method 101, the machine learning system 201 may associate the functional biomarker measurements with the known cancer statuses. The known cancer statuses provided to the machine learning algorithm may be, for example, a simple diagnosis (e.g., the patient was confirmed positive for cancer), a prognosis (i.e., good, fair or poor), treatment selection, mortality, cancer severity, known response to a treatment (i.e., effectiveness of treatment), and quality of life (e.g., changes in quality of live over the time span beginning at diagnosis). Depending on the outcomes provided to the machine learning algorithm, the trained algorithm can then be used to identify patterns indicative of the various outcomes and then to determine a likelihood of a patient having an outcome, or a combination of outcomes based on the training data set.

Any machine learning algorithm may be used to analyze the data including, for example, a random forest, a support vector machine (SVM), or a boosting algorithm (e.g., adaptive boosting (AdaBoost), gradient boost method (GBM), or extreme gradient boost methods (XGBoost)), or neural networks such as H2O.

Machine learning algorithms generally are of one of the following types: (1) bagging (decrease variance), (2) boosting (decrease bias), or (3) stacking (improving predictive force). In bagging, multiple prediction models (generally of the same type) are constructed from subsets of classification data (classes and features) and then combined into a single classifier. Random Forest classifiers are of this type. In boosting, an initial prediction model is iteratively improved by examining prediction errors. AdaBoost and eXtreme Gradient Boosting are of this type. In stacking models, multiple prediction models (generally of different types) are combined to form the final classifier. These methods are called ensemble methods. The fundamental or starting methods in the ensemble methods are often decision trees. Decision trees are non-parametric supervised learning methods that use simple decision rules to infer the classification from the features in the data. They have some advantages in that they are simple to understand and can be visualized as a tree starting at the root (usually a single node) and repeatedly branch to the leaves (multiple nodes) that are associated with the classification.

In some embodiments, method 101 and system 301 of the invention use a machine learning system 201 that uses a random forest 209. Random forests use decision tree learning, where a model is built that predicts the value of a target variable based on several input variables. Decision trees can generally be divided into two types. In classification trees, target variables take a finite set of values, or classes, whereas in regression trees, the target variable can take continuous values, such as real numbers. Examples of decision tree learning include classification trees, regression trees, boosted trees, bootstrap aggregated trees, random forests, and rotation forests. In decision trees, decisions are made sequentially at a series of nodes, which correspond to input variables. Random forests include multiple decision trees to improve the accuracy of predictions. See Breiman, 2001, Random Forests, Machine Learning 45:5-32, incorporated by reference. In random forests, bootstrap aggregating or bagging is used to average predictions by multiple trees that are given different sets of training data. In addition, a random subset of features is selected at each split in the learning process, which reduces spurious correlations that can results from the presence of individual features that are strong predictors for the response variable.

SVMs can be used for classification and regression. When used for classification of new data into one of two categories, such as having a disease or not having a disease, a SVM creates a hyperplane in multidimensional space that separates data points into one category or the other. Although the original problem may be expressed in terms that require only finite dimensional space, linear separation of data between categories may not be possible in finite dimensional space. Consequently, multidimensional space is selected to allow construction of hyperplanes that afford clean separation of data points. See Press, W. H. et al., Section 16.5. Support Vector Machines. Numerical Recipes: The Art of Scientific Computing (3rd ed.). New York: Cambridge University (2007), incorporated herein by reference. SVMs can also be used in support vector clustering. See Ben-Hur, 2001, Support Vector Clustering, J Mach Learning Res 2:125-137, incorporated by reference.

Boosting algorithms are machine learning ensemble meta-algorithms for reducing bias and variance. Boosting is focused on turning weak learners into strong learners where a weak learner is defined to be a classifier which is only slightly correlated with the true classification while a strong learner is a classifier that is well-correlated with the true classification. Boosting algorithms consist of iteratively learning weak classifiers with respect to a distribution and adding them to a final strong classifier. The added classifiers are typically weighted in based on their accuracy. Boosting algorithms include AdaBoost, gradient boosting, and XGBoost. See Freund, 1997, A decision-theoretic generalization of on-line learning and an application to boosting, J Comp Sys Sci 55:119; and Chen, 2016, XGBoost: A Scalable Tree Boosting System, arXiv: 1603.02754, both incorporated by reference.

Neural networks, modeled on the human brain, allow for processing of information and machine learning. Neural networks include nodes that mimic the function of individual neurons, and the nodes are organized into layers. Neural networks include an input layer, an output layer, and one or more hidden layers that define connections from the input layer to the output layer. Systems and methods of the invention may include any neural network that facilitates machine learning. The system may include a known neural network architecture, such as GoogLeNet (Szegedy, et al. Going deeper with convolutions, in CVPR 2015, 2015); AlexNet (Krizhevsky, et al. Imagenet classification with deep convolutional neural networks, in Pereira, et al. Eds., Advances in Neural Information Processing Systems 25, pages 1097-3105, Curran Associates, Inc., 2012); VGG16 (Simonyan & Zisserman, Very deep convolutional networks for large-scale image recognition, CoRR, abs/3409.1556, 2014); or FaceNet (Wang et al., Face Search at Scale: 80 Million Gallery, 2015), each of the aforementioned references are incorporated by reference.

Deep learning neural networks (also known as deep structured learning, hierarchical learning or deep machine learning) include a class of machine learning operations that use a cascade of many layers of nonlinear processing units for feature extraction and transformation. Each successive layer uses the output from the previous layer as input. The algorithms may be supervised or unsupervised and applications include pattern analysis (unsupervised) and classification (supervised). Certain embodiments are based on unsupervised learning of multiple levels of features or representations of the data. Higher level features are derived from lower level features to form a hierarchical representation. Those features are preferably represented within nodes as feature vectors. Deep learning by the neural network includes learning multiple levels of representations that correspond to different levels of abstraction; the levels form a hierarchy of concepts. In some embodiments, the neural network includes at least 5 and preferably more than ten hidden layers. The many layers between the input and the output allow the system to operate via multiple processing layers.

Deep learning is part of a broader family of machine learning methods based on learning representations of data. An observation can be represented in many ways such as a vector of intensity values per pixel, or in a more abstract way as a set of edges, regions of particular shape, etc. Those features are represented at nodes in the network. Preferably, each feature is structured as a feature vector, a multi-dimensional vector of numerical features that represent some object. The feature provides a numerical representation of objects, since such representations facilitate processing and statistical analysis. Feature vectors are similar to the vectors of explanatory variables used in statistical procedures such as linear regression. Feature vectors are often combined with weights using a dot product in order to construct a linear predictor function that is used to determine a score for making a prediction.

The vector space associated with those vectors may be referred to as the feature space. In order to reduce the dimensionality of the feature space, dimensionality reduction may be employed. Higher-level features can be obtained from already available features and added to the feature vector, in a process referred to as feature construction. Feature construction is the application of a set of constructive operators to a set of existing features resulting in construction of new features.

Within the network, nodes are connected in layers, and signals travel from the input layer to the output layer. In certain embodiments, each node in the input layer corresponds to a respective one of the features from the training data. The nodes of the hidden layer are calculated as a function of a bias term and a weighted sum of the nodes of the input layer, where a respective weight is assigned to each connection between a node of the input layer and a node in the hidden layer. The bias term and the weights between the input layer and the hidden layer are learned autonomously in the training of the neural network. The network may include thousands or millions of nodes and connections. Typically, the signals and state of artificial neurons are real numbers, typically between 0 and 1. Optionally, there may be a threshold function or limiting function on each connection and on the unit itself, such that the signal must surpass the limit before propagating. Back propagation is the use of forward stimulation to modify connection weights, and is sometimes done to train the network using known correct outputs. See WO 2016/182551, U.S. Pub. 2016/0174902, U.S. Pat. No. 8,639,043, and U.S. Pub. 2017/0053398, each incorporated by reference.

In some embodiments, the datasets are used to cluster a training set. Particular exemplary clustering techniques that can be used in the present invention include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

Bayesian networks are probabilistic graphical models that represent a set of random variables and their conditional dependencies via directed acyclic graphs (DAGs). The DAGs have nodes that represent random variables that may be observable quantities, latent variables, unknown parameters or hypotheses. Edges represent conditional dependencies; nodes that are not connected represent variables that are conditionally independent of each other. Each node is associated with a probability function that takes, as input, a particular set of values for the node's parent variables, and gives (as output) the probability (or probability distribution, if applicable) of the variable represented by the node.

Regression analysis is a statistical process for estimating the relationships among variables such as features and outcomes. It includes techniques for modeling and analyzing relationships between a multiple variables. Specifically, regression analysis focuses on changes in a dependent variable in response to changes in single independent variables. Regression analysis can be used to estimate the conditional expectation of the dependent variable given the independent variables. The variation of the dependent variable may be characterized around a regression function and described by a probability distribution. Parameters of the regression model may be estimated using, for example, least squares methods, Bayesian methods, percentage regression, least absolute deviations, nonparametric regression, or distance metric learning.

In some embodiments, the machine learning system may learn in a supervised or unsupervised fashion. A machine learning system that learns in an unsupervised fashion may be referred to as an autonomous machine learning system. While other versions are within the scope of the invention, an autonomous machine learning system can employ periods of both supervised and unsupervised learning. As such, in one embodiment, the random forest 209 may be operated autonomously and may include periods of both supervised and unsupervised learning. See Criminisi, 2012, Decision Forests: A unified framework for classification, regression, density estimation, manifold learning and semi-supervised learning, Foundations and Trends in Computer Graphics and Vision 7(2-3):81-227, incorporated by reference. Thus in some embodiments, the autonomous machine learning system 201 comprises a random forest 209. In some embodiments, the autonomous machine learning system 201 discovers the associations via operations that include at least a period of unsupervised learning. In preferred embodiments, the discovered associations including patterns of association between functional biomarker measurement data and at least one other data source such as RNA expression levels.

Where the algorithm is trained on treatment outcomes, it can then be used to predict a patient's responsiveness to various cancer-specific therapies. Accordingly, methods may include recommending a treatment based in part on the prediction where a certain treatment will only be recommended for patients likely to respond thereto. In certain embodiments, the recommended treatment may be provided in a report for the patient or a treating physician. In some embodiments, the treatment may be prescribed for the patient or administered to the patient.

The method 101 and system 301 may be provided with patient data from an individual. That is, the machine learning system 201 has learned from the training data set patterns or associations that are predictive of disease. The system 201 may then be applied to an individual to predicting a cancer state for the individual when the patient data presents one or more of the discovered associations. Upon detecting that association among the patient data for the individual, the machine learning system further generates a report providing information related to the cancer evaluation

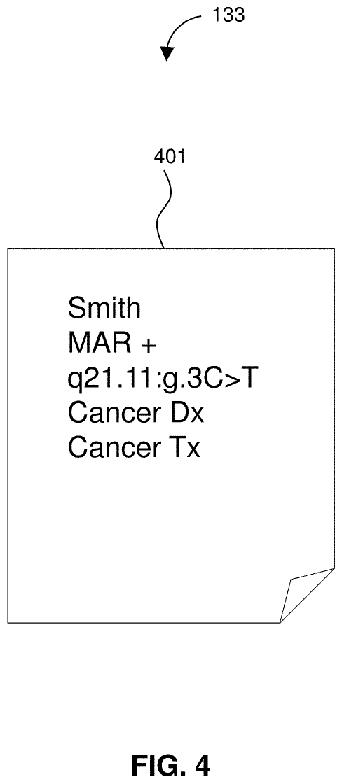

FIG. 4 shows a report 401 as may be provided by systems and methods of the invention. A report 401 may take any suitable format. For example, in certain embodiments, the report is an electronic document that is both human-readable and machine-readable, such as a PDF with text-searchable fields or an XML document shared within a system that applies style sheets for display. The report 401 may include information identifying a patient, information related to the cancer evaluation, including, but not limited to, specific data associated with a sample having undergone testing, whether the test is positive for cancer, a determination of a stage or progression of cancer, and personalized treatment tailored to an individual patient's cancer, including treatment options, depth of response, known treatment efficacies, and progression free survival. The report 401 may further provide predictive information, such as a prediction of risk of cancer for this patient in the future. As such, the report provided by systems and method of the present invention allows the health professional to initiate additional tests and begin treatment interventions far earlier than would otherwise have been possible.

Methods of the present invention further include a step of providing patient data from an individual and predicting, by the machine learning system, a cancer state for the individual when the patient data presents one or more of the discovered associations. The patient data may include functional and/or genetic data obtained from one or more assays performed on a biological sample of a patient either known to be, or suspected of being, cancerous.

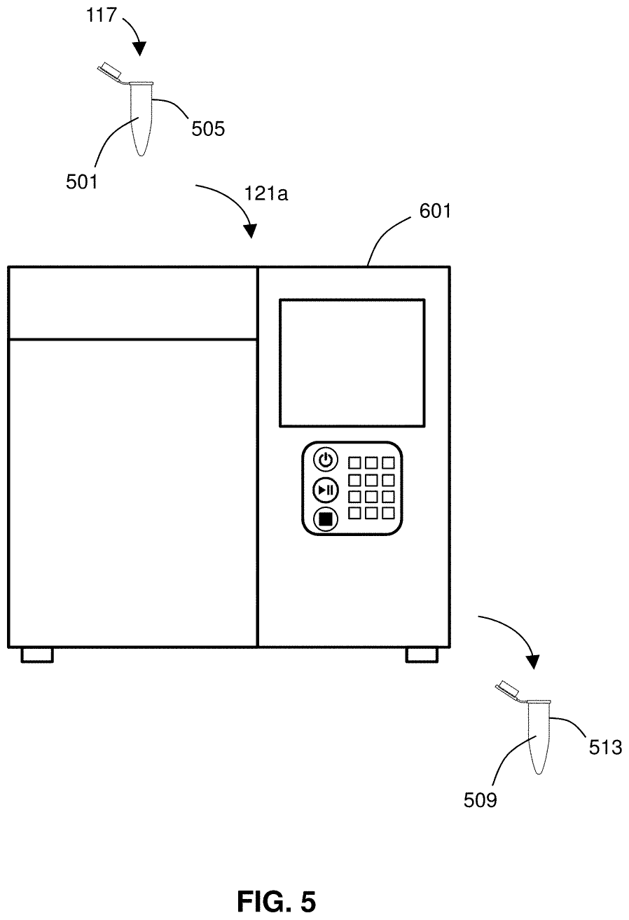

FIG. 5 shows measurement of biophysical properties of a single cell. A sample 501 may be provided within a suitable container 505, wherein the sample 501 includes one or more live cells including at least one of a cancer cell and a cancer-related immune cell obtained 117 from a patient known to have, or suspected of having, cancer. For example, in some embodiments, samples may be collected and stored in their own container, such as a centrifuge tube such as the 1.5 mL micro-centrifuge tube sold under the trademark EPPENDORF FLEX-TUBES by Eppendorf, Inc. (Enfield, Conn.).

The one or more live cells are isolated from a biological sample of a patient known to have, or suspected of having, cancer. A biological sample may include a human tissue or bodily fluid and may be collected in any clinically acceptable manner. For example, the sample may include a fine needle aspirate or a biopsy from a tissue known to be, or suspected of being, cancerous. The sample may include a bodily fluid from a patient either known to include, or suspected of including, cancer cells or cancer-related cells (i.e., immune cells).

A tissue may include a mass of connected cells and/or extracellular matrix material, e.g. skin tissue, hair, nails, nasal passage tissue, CNS tissue, neural tissue, eye tissue, liver tissue, kidney tissue, placental tissue, mammary gland tissue, placental tissue, mammary gland tissue, gastrointestinal tissue, musculoskeletal tissue, genitourinary tissue, bone marrow, and the like, derived from, for example, a human or other mammal and includes the connecting material and the liquid material in association with the cells and/or tissues.

A body fluid may be a liquid material derived from, for example, a human or other mammal. Such body fluids include, but are not limited to, mucous, blood, plasma, serum, serum derivatives, bile, blood, maternal blood, phlegm, saliva, sputum, sweat, amniotic fluid, menstrual fluid, mammary fluid, follicular fluid of the ovary, fallopian tube fluid, peritoneal fluid, urine, semen, and cerebrospinal fluid (CSF), such as lumbar or ventricular CS. A sample also may be media containing cells or biological material. A sample may also be a blood clot, for example, a blood clot that has been obtained from whole blood after the serum has been removed. In certain embodiments, the sample is blood, saliva, or semen collected from the subject.

The isolation of the one or more live cells from the biological sample may be performed via any known isolation techniques and methods for maintaining a viable collection of cells, which may include one or cancer and/or cancer-related immune cells (e.g., lymphocytes includes T-cells and/or B-cells). For example, if the sample is a tissue sample from a tumor or growth suspected of being cancerous, the tissue sample may undergo any known cell isolation, separation, or dissociation techniques which may involve physical methods (i.e., use of mechanical force to break apart cellular adhesions) and/or reagent-based methods (i.e., use of fluid mediums to break apart cellular adhesions). For example, in one embodiment, a tissue sample (i.e., a fine needle aspirate from a tumor) may be disaggregated to produce a suspension of individual live cells to allow for analysis of cells independently. The tissue sample may undergo initial disaggregation by way of application of a physical force alone to break the tissue sample into smaller pieces, at which point the sample may be exposed to proteolytic enzymes that digest cellular adhesion molecules and/or the underlying extracellular matrix to thereby provide single cells within a suspension. It should be noted that the reagents selected for assisting in the disaggregating step should keep the cells intact and not kill the cells.

Other methods currently used for single cell isolation include, but are not limited to, serial dilution, micromanipulation, laser capture microdissection, FACS, microfluidics, Dielectrophoretic digital sorting, manual picking, and Raman tweezers. Manual single cell picking is a method is where cells in a suspension are viewed under a microscope, and individually picked using a micropipette, while Raman tweezers is a technique where Raman spectroscopy is combined with optical tweezers, which uses a laser beam to trap, and manipulate cells. Dielectrophoretic (DEP) digital sorting method utilizes a semiconductor controlled array of electrodes in a microfluidic chip to trap single cells in DEP cages, where cell identification is ensured by the combination of fluorescent markers with image observation and delivery is ensured by the semiconductor controlled motion of DEP cages in the flow cell.

Live cells are loaded onto an instrument 601 capable of performing a first assay on the live cells to thereby measure 121a at least a first functional biomarker of one or more live cells. The instrument 601 measures a functional biomarker in the one or more live cells, such as single-cell biophysical properties, including, but not limited to, mass, growth rate, and mass accumulation of an individual living cell. The initial assay may generally be performed with an instrument 601 comprising a suspended microchannel resonator (SMR). The SMR may be used to precisely measure biophysical properties, such as mass and mass changes, of a single cell flowing therethrough. The mass change may be mass accumulation rate (MAR). When used with cancer cells, those changes provide a functional, universal biomarker by which medical professionals (e.g., oncologists) may monitor the progression of a cancer and determine how cancer cells respond to therapies.

The SMR may comprise an exquisitely sensitive scale that measures small changes in mass of a single cell. When cancer cells respond to cancer drugs, the cells begin the process of dying by changing mass within hours. The SMR can detect this minor weight change. That speed and sensitivity allow the SMR to detect a cancer cell's response to a cancer drug while the cell is still living. Upon flowing the live cells through the SMR, a functional biomarker, such as mass or MAR, in the one or more live cells is obtained. MAR measurements characterize heterogeneity in cell growth across cancer cell lines. Individual live cells are able to pass through the SMR, wherein each cell is weighed multiple times over a defined interval. The SMR includes multiple sensors that are fluidically connected, such as in series, and separated by delay channels. Such a design enables a stream of cells to flow through the SMR such that different sensors can concurrently weigh flowing cells in the stream, revealing single-cell MARs. The SMR is configured to provide real-time, high-throughput monitoring of mass change for the cells flowing therethrough. Therefore, the biophysical properties, including mass and/or mass changes (e.g., MAR), of a single cell can be measured. Such data can be stored and used in subsequent analysis steps, as will be described in greater detail herein.

Upon passing through the instrument 601, single cells remain viable and can be isolated downstream from the instrument 601 and are available to undergo the subsequent assays. As shown, a sample 509 of the one or more live cells having undergone the first assay (i.e., passing through the instrument 601) are collected in a suitable container 513 and are then available to undergo a second assay.

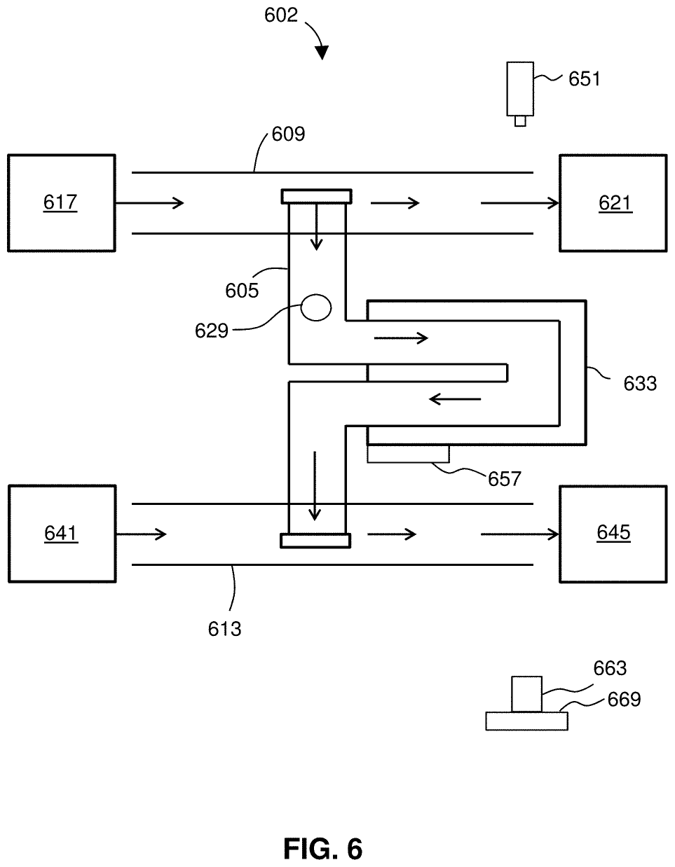

FIG. 6 shows a suspended microchannel resonator (SMR) device 602 of the disclosure. The SMR device 602 includes a microchannel 605 that runs through a cantilever 633, which is suspended between an upper bypass channel 609 and a lower bypass channel 613. Having the two bypass channels allows for decreased flow resistance and accommodates the flow rate through the microchannel 605. Sample eluate 617 flows through the upper bypass channel 609, wherein a portion of the eluate 617 collects in the upper bypass channel collection reservoir 621. A portion of the eluate 617 including at least one live cell 629 flows through the suspended microchannel 605. The flow rate through the suspended microchannel 605 is determined by the pressure difference between its inlet and outlet. Since the flow cross section of the suspended microchannel is about 70 times smaller than that of the bypass channels, the linear flow rate can be much faster in the suspended microchannel than in the bypass channel, even though the pressure difference across the suspended microchannel is small. Therefore, at any given time, it is assumed that the SMR is measuring the eluate that is present at the inlet of the suspended microchannel. The sample includes a live cell or material with cell-like properties.

The cell 629 flows through the suspended microchannel 605. The suspended microchannel 605 extends through a cantilever 633 which sits between a light source 651 and a photodetector 663 connected to a chip 669 such as a field programmable gate array (FPGA). The cantilever is operated on by an actuator, or resonator 657. The resonator 657 may be a piezo-ceramic actuator seated underneath the cantilever 633 for actuation. The cell 629 flows from the upper bypass channel 609 to the inlet of the suspended microchannel 605, through the suspended microchannel 605, and to the outlet of the suspended microchannel 605 toward the lower bypass channel 613. A buffer 641 flows through the lower bypass channel towards a lower bypass channel collection reservoir 645. After the cell 629 is introduced to the lower bypass channel 613, the cell 629 is collected in the lower bypass collection reservoir 645.

In some embodiments, the instrument 601 comprises an array of SMRs with a fluidic channel passing therethrough.

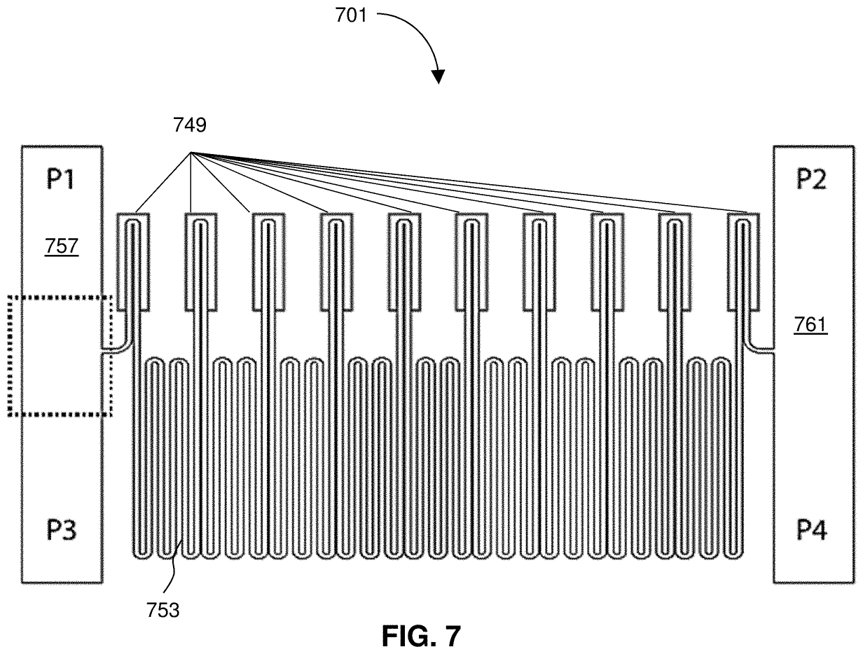

FIG. 7 shows a serial suspended microchannel resonator (sSMR) array 701, made up of an array of SMRs. An instrument that includes an sSMR array is useful for direct measurement of biophysical properties of single cells flowing therethrough. The sSMR includes a plurality of cantilevers 749 and a plurality of delay channels 753. Cells from the first bypass channel 757 through the cantilevers 749 and delay channels 753 to the second bypass channel 761. Pressure differences in the first bypass channel 757 are indicated by P1 and P2, and pressure differences in the second bypass channel 761 are indicated by P3 and P4.

Instruments 601 of the disclosure can make sensitive and precise measurements of mass or change in mass through the use of an sSMR array 701. The instruments use a structure such as a cantilever that contains a fluidic microchannel. Living cells are flowed through the structure, which is resonated and its frequency of resonation is measured. The frequency at which a structure resonates is dependent on its mass and by measuring the frequency of at which the cantilever resonates, the instrument can compute a mass, or change in mass, of a living cell in the fluidic microchannel. By flowing the isolated living cells from the tissue sample through such devices, one may observe the functions of those cells, such as whether they are growing and accumulating mass or not. The mass accumulation or rate of mass accumulation can be related to clinically important property such as the presence of cancer cells or the efficacy of a therapeutic on cancer cells.

Methods for measuring single-cell growth are based on resonating micromechanical structures. The methods exploit the fact that a micromechanical resonator's natural frequency depends on its mass. Adding cells to a resonator alters the resonator's mass and causes a measurable change in resonant frequency. Suspended microchannel resonators (SMRs) include a sealed microfluidic channel that runs through the interior of a cantilever resonator. The cantilever itself may be housed in an on-chip vacuum cavity, reducing damping and improving frequency (and thus mass) resolution. As a cell in suspension flows through the interior of the cantilever, it transiently changes the cantilever's resonant frequency in proportion to the cell's buoyant mass (the cell's mass minus the fluid mass it displaces). SMRs weigh single mammalian cells with a resolution of 0.05 pg (0.1% of a cell's buoyant mass) or better. The sSMR array 701 includes an array of SMRs fluidically connected in series and separated by “delay” channels between each cantilever 349. The delay channels give the cell time to grow as it flows between cantilevers.

Devices may be fabricated as described in Lee, 2011, Suspended microchannel resonators, Lab Chip 11:645 and/or Burg, 2007, Weighing of biomolecules, Nature 446:1066-1069, both incorporated by reference. Large-channel devices (e.g., useful for PBMC measurements) may have cantilever interior channels of 15 by 20 μm in cross-section, and delay channels 20 by 30 μm in cross-section. Small-channel devices (useful for a wide variety of cell types) may have cantilever channels 3 by 5 μm in cross-section, and delay channels 4 by 15 μm in cross-section. The tips of the cantilevers in the array may be aligned so that a single line-shaped laser beam can be used for optical-lever readout. The cantilevers may be arrayed such that the shortest (and therefore most sensitive) cantilevers are at the ends of the array. Before use, the device may be cleaned with piranha (3:1 sulfuric acid to 50% hydrogen peroxide) and the channel walls may be passivated with polyethylene glycol (PEG) grafted onto poly-L-lysine. In some embodiments, a piezo-ceramic actuator seated underneath the device is used for actuation.

The instrument 601 may include low-noise photodetector, Wheatstone bridge-based amplifier (for piezo-resistor readout), and high-current piezo-ceramic driver. To avoid the effects of optical interference between signals from different cantilevers (producing harmonics at the difference frequency), the instrument may include a low-coherence-length light source (675 nm super-luminescent diode, 7 nm full-width half maximum spectral width) as an optical lever. After the custom photodetector converts the optical signal to a voltage signal, that signal is fed into an FPGA board, in which an FPGA implements twelve parallel second-order phase-locked loops which each both demodulate and drive a single cantilever. The FPGA may on a DE2-115 development board operating on a 100 MHz clock with I/O provided via a high-speed AD/DA card operating 14-bit analog-to-digital and digital-to-analog converters at 100 MHz.

To operate all cantilevers in the array, the resonator array transfer function is first measured by sweeping the driving frequency and recording the amplitude and phase of the array response. Parameters for each phase-locked loop (PLL) are calculated such that each cantilever-PLL feedback loop has a 50 or 100 Hz FM-signal bandwidth. The phase-delay for each PLL may be adjusted to maximize the cantilever vibration amplitude. The FM-signal transfer function may be measured for each cantilever-PLL feedback loop to confirm sufficient measurement bandwidth (in case of errors in setting the parameters). That transfer function relates the measured cantilever-PLL oscillation frequency to a cantilever's time-dependent intrinsic resonant frequency. Frequency data for each cantilever are collected at 500 Hz, and may be transmitted from the FPGA to a computer. The device may be placed on a copper heat sink/source connected to a heated water bath, maintained at 37 degrees C. The sample is loaded into the device from vials pressurized under air or air with 5% CO2 through 0.009 inch inner-diameter fluorinated ethylene propylene (FEP) tubing. The pressurized vials may be seated in a temperature-controlled sample-holder throughout the measurement. FEP tubing allows the device to be flushed with piranha solution for cleaning, as piranha will damage most non-fluorinated plastics. To measure a sample of cells, the device may initially flushed with filtered media, and then the sample may be flushed into one bypass channel. On large-channel devices, between one and two psi may be applied across the entire array, yielding flow rates on the order of 0.5 nL/s (the array's calculated fluidic resistance is approximately 3×10\hat{ }16 Pa/(m3/s). For small-channel devices, 4-5 psi may be applied across the array, yielding flow rates around 0.1 nL/s. Additionally, every several minutes new sample may be flushed into the input bypass channel to prevent particles and cells from settling in the tubing and device. Between experiments, devices may be cleaned with filtered 10% bleach or piranha solution.

For the data analysis, the recorded frequency signals from each cantilever are rescaled by applying a rough correction for the different sensitivities of the cantilevers. Cantilevers differing in only their lengths should have mass sensitivities proportional to their resonant frequencies to the power three-halves. Therefore each frequency signal is divided by its carrier frequency to the power three-halves such that the signals are of similar magnitude. To detect peaks, the data are filtered with a low pass filter, followed by a nonlinear high pass filter (subtracting the results of a moving quantile filter from the data). Peak locations are found as local minima that occur below a user-defined threshold. After finding the peak locations, the peak heights may be estimated by fitting the surrounding baseline signal (to account for a possible slope in the baseline that was not rejected by the high pass filter), fitting the region surrounding the local minima with a fourth-order polynomial, and finding the maximum difference between the predicted baseline and the local minima polynomial fit. Identifying the peaks corresponding to calibration particles allows one to estimate the mass sensitivity for each cantilever, such that the modal mass for the particles is equal to the expected modal mass. Peaks at different cantilevers that originate from the same cell are matched up to extract single-cell growth information. The serial SMR array and can measure live cells.

Certain embodiments include devices with piezo-resistors doped into the base of each cantilever, which are wired in parallel and their combined resistance measured via a Wheatstone bridge-based amplifier. The resulting deflection signal, which consists of the sum of k signals from the cantilever array, goes to an array of k phase-locked loops (PLLs) where each PLL locks to the unique resonant frequency of a single cantilever. Therefore there is a one to one pairing between cantilevers and PLLs. Each PLL determines its assigned cantilever's resonant frequency by demodulating its deflection signal and then generates a sinusoidal drive signal at that frequency. The drive signals from each PLL are then summed and used to drive a single piezo actuator positioned directly underneath the chip, completing the feedback loop. Each PLL is configured such that it will track its cantilever's resonant frequency with a bandwidth of 50 or 100 Hz. After acquiring the frequency signals for each cantilever, the signals are converted to mass units via each cantilever's sensitivity (Hz/pg), which is known precisely.

Various embodiments of SMR and sSMR instruments, as well as methods of use, include those instruments/devices manufactured by Innovative Micro Technology (Santa Barbara, Calif.) and described in U.S. Pat. Nos. 8,418,535 and 9,132,294, the contents of each of which are hereby incorporated by reference in their entirety.

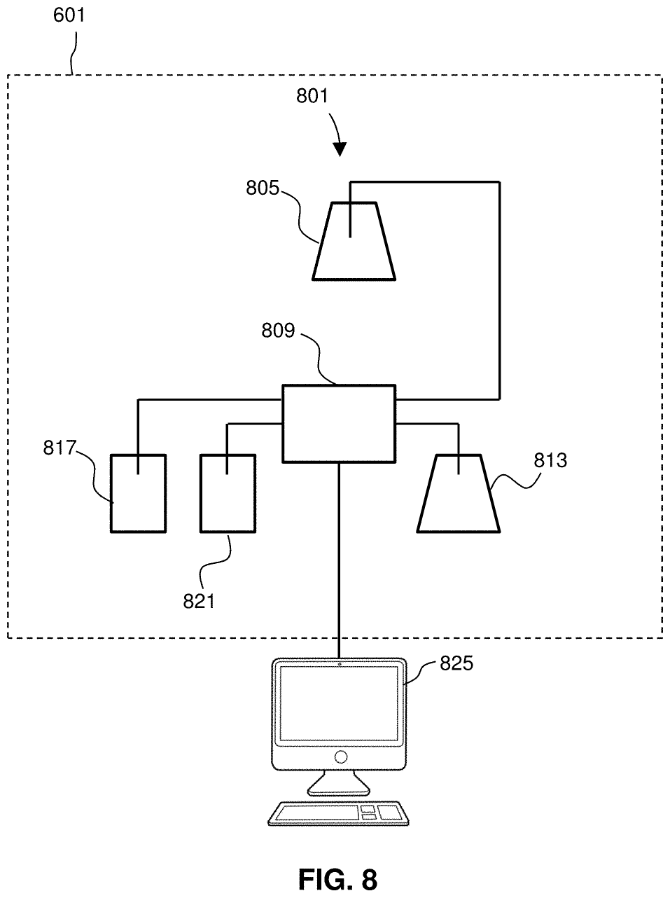

FIG. 8 shows a schematic diagram of an SMR detection system 801. As shown, a sample 805 (i.e., one or more live cells provided in a fluid medium) may be introduced to the SMR 809 of an instrument 601. As shown, the sample 805 and a buffer solution 813 may be provided to the SMR. The system 801 further includes an upper bypass channel collection outlet/reservoir 817 and lower bypass channel collection outlet/reservoir 821. The SMR 809 is configured to measure a functional biomarker of one or more live cells 805 flowing therethrough, such as density or mass of the sample, and transmit such measurements to a computer 825 that is communicatively coupled to the SMR 809, specifically communicatively coupled to the instrument 601. The computer 825 may be used for analysis and reporting of results. In some embodiments, a system for the functional biomarker measurement instrument may include additional analytical techniques, as will be described in greater detail herein. The computer 825 may further comprise a server and storage. Any of the elements in the SMR detection system 801 may interoperate via a network. The SMR 809 may include its own on-board computer. The computer 825 may include one or more processors and memory as well as an input/output mechanism.

Upon passing through the instrument 601, namely the exemplary flow path of a suspended microchannel or the flow path of the sSMR array 701, the cells remain viable and can be isolated downstream from the instrument 601 and are available to undergo the subsequent assays. The method further includes performing one or more additional assays on the live cells, either concurrently with the initial assay, or downstream from the first assay, to obtain further data associated with the live cells, such as additional functional data and/or genomic data.

It should be noted that methods of the disclosure include performing one or more additional assays on the live cells, either concurrently with the first assay, or downstream from the first assay, to obtain further functional or genetic data. In some embodiments, the second assay is performed on the live cells having undergone the first assay, which allows for data obtained from the first and second assays to be linked at a single-cell level, as opposed to a population level.

The one or more additional assays allow for single-cell analysis, including, for example, genome sequencing, single-cell transcriptomics, single-cell proteomics, and single-cell metabolomics.

Genome sequencing is generally the process of determining the order of nucleotides in DNA. It includes any method or technology that is used to determine the order of the four bases: adenine, guanine, cytosine, and thymine. Single cell DNA genome sequencing involves isolating a single cell, performing whole genome amplification (WGA), constructing sequencing libraries, and then sequencing the DNA using a next-generation sequencer (e.g., Illumina, Ion Torrent, etc.). Single cell genome sequencing is particularly of interest in the field of cancer study, as cancer cells are constantly mutating and it is of great interest to observer how cancers evolve at the genetic level. For example, single cell genome sequencing allowing for patterns of somatic mutations and copy number aberration to be observed.

Single-cell transcriptomics examines the gene expression level of individual cells in a given population by simultaneously measuring the messenger RNA (mRNA) concentration of hundreds to thousands of genes.

The purpose of single cell transcriptomics is to determine what genes are being expressed in each cell. The transcriptome is often used to quantify the gene expression instead of the proteome because of the difficulty currently associated with amplifying protein levels. Single-cell transcriptomics uses sequencing techniques similar to single cell genomics or direct detection using fluorescence in situ hybridization. The first step in quantifying the transcriptome is to convert RNA to cDNA using reverse transcriptase so that the contents of the cell can be sequenced using NGS methods, similar to what is done in single-cell genomics. Once converted, cDNA undergoes whole genome amplification (WGA), and then sequencing is performed. Alternatively, fluorescent compounds attached to RNA hybridization probes may be used to identify specific sequences and sequential application of different RNA probes will build up a comprehensive transcriptome.

Single cell transcriptomics can be used for various studies, such as, for example, gene dynamics, RNA splicing, and cell typing. Gene dynamics are usually studied to determine what changes in gene expression effect different cell characteristics. For example, this type of transcriptomic analysis has often been used to study embryonic development. RNA splicing studies are focused on understanding the regulation of different transcript isoforms. Single cell transcriptomics has also been used for cell typing, where the genes expressed in a cell are used to identify types of cells.

Single-cell proteomics is the study of proteomes (the entire complement of proteins that is or can be expressed by a cell, tissue, or organism) and their functions. The purpose of studying the proteome is to better understand the activity of cells at the single cells level. Since proteins are responsible for determining how the cell acts, understanding the proteome of single cell gives the best understanding of how a cell operates, and how gene expression changes in a cell due to different environmental stimuli. Although transcriptomics has the same purpose as proteomics it is not as accurate at determining gene expression in cells as it does not take into account post-transcriptional regulation.

There are three major approaches to single-cell proteomics: antibody based methods; fluorescent protein based methods; and mass-spectroscopy based methods. The antibody based methods use designed antibodies to bind to proteins of interest. These antibodies can be bound to fluorescent molecules such as quantum dots or isotopes that can be resolved by mass spectrometry. Since different colored quantum dots or different isotopes are attached to different antibodies it is possible to identify multiple different proteins in a single cell. Rare metal isotopes attached to antibodies, not normally found in cells or tissues, can be detected by mass spectrometry for simultaneous and sensitive identification of proteins. Another antibody based method converts protein levels to DNA levels. The conversion to DNA makes it possible to amplify protein levels and use NGS to quantify proteins. To do this, two antibodies are designed for each protein needed to be quantified. The two antibodies are then modified to have single stranded DNA connected to them that are complimentary. When the two antibodies bind to a protein the complimentary strands will anneal and produce a double stranded piece of DNA that can then be amplified using PCR. Each pair of antibodies designed for one protein is tagged with a different DNA sequence. The DNA amplified from PCR can then be sequenced, and the protein levels quantified.

In mass spectroscopy-based proteomics, there are three major steps needed for peptide identification: sample preparation; separation of peptides; and identification of peptides. Several groups have focused on oocytes or very early cleavage-stage cells since these cells are unusually large and provide enough material for analysis. Another approach, single cell proteomics by mass spectrometry (SCoPE-MS) has quantified thousands of proteins in mammalian cells with typical cell sizes (diameter of 10-15 μm) by combining carrier-cells and single-cell barcoding. Multiple methods exist to isolate the peptides for analysis. These include using filter aided sample preparation, the use of magnetic beads, or using a series of reagents and centrifuging steps. The separation of differently sized proteins can be accomplished by using capillary electrophoresis (CE) or liquid chromatograph (LC) (using liquid chromatography with mass spectroscopy is also known as LC-MS). This step gives order to the peptides before quantification using tandem mass-spectroscopy (MS/MS). The major difference between quantification methods is some use labels on the peptides such as tandem mass tags (TMT) or dimethyl labels which are used to identify which cell a certain protein came from (proteins coming from each cell have a different label) while others use not labels (quantify cells individually). The mass spectroscopy data is then analyzed by running data through databases that convert the information about peptides identified to quantification of protein levels. These methods are very similar to those used to quantify the proteome of bulk cells, with modifications to accommodate the very small sample volume. Improvements in sample preparation, mass-spec methods and data analysis can increase the sensitivity and throughput by orders of magnitude.

Single-cell metabolomics is study of chemical processes involving metabolites, the small molecule intermediates and products of metabolism, within cells. In particular, the purpose of single cell metabolomics is to gain a better understanding at the molecular level of major biological topics such as: cancer, stem cells, aging, as well as the development of drug resistance. In general the focus of metabolomics is mostly on understanding how cells deal with environmental stresses at the molecular level, and to give a more dynamic understanding of cellular functions. Accordingly, single cell metabolomics involves the study of a metabolome, which represents the complete set of metabolites in a biological cell, which are the end products of cellular processes. As generally understood, mRNA gene expression data and proteomic analyses reveal the set of gene products being produced in the cell, data that represents one aspect of cellular function. Conversely, metabolic profiling can give an instantaneous snapshot of the physiology of that cell, and thus, metabolomics provides a direct functional readout of the physiological state of an organism.

There are four major methods used to quantify the metabolome of single cells: fluorescence-based detection, fluorescence biosensors, FRET biosensors, and mass spectroscopy. The fluorescence-based detection, fluorescence biosensors, and FRET biosensors methods each use fluorescence microscopy to detect molecules in a cell. Such assays use small fluorescent tags attached to molecules of interest. However, it has been found that use of fluorescent tags may be too invasive for single cell metabolomics, and alters the activity of the metabolites. As such, the current solution to this problem is to use fluorescent proteins which will act as metabolite detectors, fluorescing whenever they bind to a metabolite of interest.

Mass spectroscopy is becoming the most frequently used method for single cell metabolomics, as there is no need to develop fluorescent proteins for all molecules of interest, and it is capable of detecting metabolites in the femtomole range. Similar to the methods discussed in proteomics, there has also been success in combining mass spectroscopy with separation techniques such as capillary electrophoresis to quantify metabolites. Another method utilizes capillary microsampling combined with mass spectrometry and ion mobility separation, which has been demonstrated to enhance the molecular coverage and ion separation for single cell metabolomics.

Yet still, in other embodiments, the one or more additional assays may include flow cytometry to analyze physical and/or chemical characteristics of the one or more cells, including the detection of biomarkers. For example, a flow cytometer may be used to detect and measure chemical characteristics of cells by suspending the cells in a fluid, injecting the cells in the instrument, and flowing one cell at a time through a laser. The fluorescence can be measured to determine various properties of single particles, which are usually cells. Up to thousands of particles per second can be analyzed as they pass through the liquid stream. Examples of the properties measured include the particle's relative granularity, size and fluorescence intensity as well as its internal complexity. An optical-to-electronic coupling system is used to record the way in which the particle emits fluorescence and scatters incident light from the laser. Any suitable instrument may be used including for example one of the cell-sorting flow cytometry instruments sold under the trademarks FACSARIAIII by BD Biosciences, MOFLO XDP sold by Beckman Coulter, S3E sold by Bio-Rad, or VIVA G1 sold by Cytonome. For example, certain embodiments may use the cell sorting instrument sold under the trademark S3E cell sorter by Bio-Rad (Hercules, Calif.).

Accordingly, in one embodiment, the second assay may include sequencing nucleic acid from the one or more live cells having undergone the first assay to produce sequence data. In order to perform nucleic acid sequencing, methods of the disclosure further include extracting nucleic acid from the one or more live cells having undergone the first analysis for a downstream sequencing step.

Isolation, extraction or derivation of genomic nucleic acids may be performed by methods known in the art. Isolating nucleic acid from a biological sample generally includes treating a biological sample in such a manner that genomic nucleic acids present in the sample are extracted and made available for analysis. Generally, nucleic acids are extracted using techniques such as those described in Green & Sambrook, 2012, Molecular Cloning: A Laboratory Manual 4 edition, Cold Spring Harbor Laboratory Press, Cold Spring Harbor, N.Y. (2028 pages), the contents of which are incorporated by reference herein. A kit may be used to extract DNA from tissues and bodily fluids and certain such kits are commercially available from, for example, BD Biosciences Clontech (Palo Alto, Calif.), Epicentre Technologies (Madison, Wis.), Gentra Systems, Inc. (Minneapolis, Minn.), and Qiagen Inc. (Valencia, Calif.). User guides that describe protocols are usually included in such kits.

It may be useful to lyse cells to isolate genomic nucleic acid. Cellular extracts can be subjected to other steps to drive nucleic acid isolation toward completion by, e.g., differential precipitation, column chromatography, extraction with organic solvents, filtration, centrifugation, others, or any combination thereof. The genomic nucleic acid may be re-suspended in a solution or buffer such as water, Tris buffers, or other buffers. In certain embodiments the genomic nucleic acid can be re-suspended in Qiagen DNA hydration solution, or other Tris-based buffer of a pH of around 7.5. Isolated nucleic acid (e.g., DNA, RNA, cDNA, etc.) may be fragmented for enhanced probe capture. Methods of nucleic acid fragmentation are known in the art and include, but are not limited to, DNase digestion, sonication, mechanical shearing, and the like. U.S. Pub 2005/0112590 provides a general overview of various methods of fragmenting known in the art. Fragmentation of nucleic acid target is discussed in U.S. Pub. 2013/0274146. The nucleic acid can also be sheared via nebulization, hydro-shearing, sonication, or others. See U.S. Pat. Nos. 6,719,449; 6,948,843; and 6,235,501.

When there is an insufficient amount of nucleic acid for analysis, a common technique used to increase the amount by amplifying the nucleic acid. Amplification refers to production of additional copies of a nucleic acid sequence and is generally carried out using polymerase chain reaction or other technologies well known in the art (e.g., Dieffenbach, PCR Primer, a Laboratory Manual, 1995, Cold Spring Harbor Press, Plainview, N.Y.). Polymerase chain reaction (PCR) refers to methods by K. B. Mullis (U.S. Pat. Nos. 4,683,195 and 4,683,202, hereby incorporated by reference) for increasing concentration of a segment of a target sequence in a mixture of genomic DNA without cloning or purification. Primers can be prepared by a variety of methods including but not limited to cloning of appropriate sequences and direct chemical synthesis using methods well known in the art (Narang et al., Methods Enzymol., 68:90 (1979); Brown et al., Methods Enzymol., 68:109 (1979)). Primers can also be obtained from commercial sources such as Operon Technologies, Amersham Pharmacia Biotech, Sigma, and Life Technologies. Amplification or sequencing adapters or barcodes, or a combination thereof, may be attached to the fragmented nucleic acid. Such molecules may be commercially obtained, such as from Integrated DNA Technologies (Coralville, Iowa). In certain embodiments, such sequences are attached to the template nucleic acid molecule with an enzyme such as a ligase. Suitable ligases include T4 DNA ligase and T4 RNA ligase, available commercially from New England Biolabs (Ipswich, Mass.). The ligation may be blunt ended or via use of complementary overhanging ends.

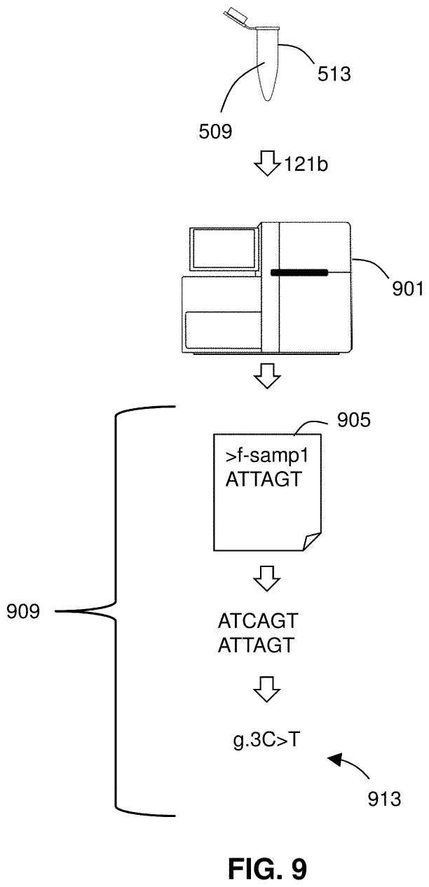

FIG. 9 diagrams a sequencing workflow according to certain embodiments. As shown, the method includes performing a second assay on the one or more live cells having undergone the first assay (i.e., sample 509 of live cells collected from the device 601), wherein the second assay includes sequencing nucleic acid from the one or more live cells (from sample 509) using a sequencing instrument 901 to produce sequence data, and, in turn, method includes obtaining a measurement 121b which includes genomic data obtained via the sequencing step.

As such, the biophysical data (i.e., growth of live cells by measuring mass or change in mass in the cells) obtained from instrument 901 and the sequence data obtained from instrument 901 can be provided to the machine learning system. The machine learning system is then able to predict a cancer status for the individual when the biophysical data and/or genetic data present one or more of the discovered associations. Upon detecting that association among the biophysical data and/or genetic data for an individual, the machine learning system further generates a report providing information related to the cancer evaluation, including, but not limited to, specific data associated with the patient sample having undergone testing, whether the test is positive for cancer, a determination of a stage or progression of cancer, and a customized treatment plan tailored to an individual patient's cancer diagnosis.

Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Separated molecules may be sequenced by sequential or single extension reactions using polymerases or ligases as well as by single or sequential differential hybridizations with libraries of probes.

A sequencing technique that can be used includes, for example, Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. Sequencing according to this technology is described in U.S. Pat. Nos. 7,960,120; 7,835,871; 7,232,656; 7,598,035; 6,911,345; 6,833,246; 6,828,100; 6,306,597; 6,210,891; U.S. Pub. 2011/0009278; U.S. Pub. 2007/0114362; U.S. Pub. 2006/0292611; and U.S. Pub. 2006/0024681, each of which is incorporated by reference in their entirety.

Sequencing produces a plurality of sequence reads 905. Sequence reads 905 generally include sequences of nucleotide data wherein read length may be associated with sequencing technology. For example, the single-molecule real-time (SMRT) sequencing technology of Pacific Bio produces reads thousands of base-pairs in length. For 454 pyrosequencing, read length may be about 700 bp in length. In some embodiments, reads are less than about 500 bases in length, or less than about 150 bases in length, or less than about 80 bases in length. In certain embodiments, reads are between about 80 and about 80 bases, e.g., about 85 bases in length. In some embodiments, these are very short reads, i.e., less than about 50 or about 30 bases in length. Sequence reads can be analyzed to detect and describe variations.

Sequence reads 905 can be stored in any suitable file format including, for example, VCF files, FASTA files or FASTQ files, as are known to those of skill in the art. In some embodiments, PCR product is pooled and sequenced (e.g., on an Illumina HiSeq 2000). Raw .bcl files are converted to qseq files using bclConverter (Illumina). FASTQ files are generated by “de-barcoding” genomic reads using the associated barcode reads; reads for which barcodes yield no exact match to an expected barcode, or contain one or more low-quality base calls, may be discarded. Reads may be stored in any suitable format such as, for example, FASTA or FASTQ format.

The sequence reads may be analyzed to identify structural abnormalities, copy number variants, microdeletions, or duplications. In some embodiments, the sequence reads 905 are analyzed to identify sub chromosomal copy number alteration or an aneuploidy.

In some embodiments, analysis of sequence reads may be used to identify small mutations such as polymorphisms or small indels, such as variant calling 909. To identify small mutations, reads may be mapped to a reference using assembly and alignment techniques known in the art or developed for use in the workflow. Various strategies for the alignment and assembly of sequence reads, including the assembly of sequence reads into contigs, are described in detail in U.S. Pat. No. 8,209,130, incorporated herein by reference. Strategies may include (i) assembling reads into contigs and aligning the contigs to a reference; (ii) aligning individual reads to the reference; (iii) assembling reads into contigs, aligning the contigs to a reference, and aligning the individual reads to the contigs; or (iv) other strategies known to be developed or known in the art. Sequence assembly can be done by methods known in the art including reference-based assemblies, de novo assemblies, assembly by alignment, or combination methods. Sequence assembly is described in U.S. Pat. Nos. 8,165,821; 7,809,509; 6,223,128; U.S. Pub. 2011/0257889; and U.S. Pub. 2009/0318310, the contents of each of which are hereby incorporated by reference in their entirety. Sequence assembly or mapping may employ assembly steps, alignment steps, or both. Assembly can be implemented, for example, by the program ‘The Short Sequence Assembly by k-mer search and 3′ read Extension’ (SSAKE), from Canada's Michael Smith Genome Sciences Centre (Vancouver, B.C., CA) (see, e.g., Warren et al., 2007, Assembling millions of short DNA sequences using SSAKE, Bioinformatics, 23:500-501). SSAKE cycles through a table of reads and searches a prefix tree for the longest possible overlap between any two sequences. SSAKE clusters reads into contigs.

Generally, read assembly and analysis will proceed through the use of one or more specialized computer programs. One read assembly program is Forge Genome Assembler, written by Darren Platt and Dirk Evers and available through the SourceForge web site maintained by Geeknet (Fairfax, Va.) (see, e.g., DiGuistini et al., 2009, De novo sequence assembly of a filamentous fungus using Sanger, 454 and Illumina sequence data, Genome Biology, 10:R94). Forge distributes its computational and memory consumption to multiple nodes, if available, and has therefore the potential to assemble large sets of reads. Forge was written in C++ using the parallel MPI library. Forge can handle mixtures of reads, e.g., Sanger, 454, and Illumina reads. Other read assembly or analysis programs include: Velvet, available through the web site of the European Bioinformatics Institute (Hinxton, UK) (Zerbino & Birney, Velvet: Algorithms for de novo short read assembly using de Bruijn graphs, Genome Research 18(5):821-829); SOAP, available through the website of Beijing Genomics Institute (Beijing, CN) or BGI Americas Corporation (Cambridge, Mass.); ABySS, from Canada's Michael Smith Genome Sciences Centre (Vancouver, B.C., CA) (Simpson et al., 2009, ABySS: A parallel assembler for short read sequence data, Genome Res., 19(6):1117-23); and Roche's GS De Novo Assembler, known as gsAssembler or Newbler (NEW assemBLER), which is designed to assemble reads from the Roche 454 sequencer (described, e.g., in Kumar & Blaxter, 2010, Comparing de novo assemblers for 454 transcriptome data, Genomics 11:571), all references incorporated by reference. Additional discussion of read assembly may be found in Li et al., 2009, The Sequence alignment/map (SAM) format and SAMtools, Bioinformatics 25:2078; Lin et al., 2008, ZOOM! Zillions Of Oligos Mapped, Bioinformatics 24:2431; Li & Durbin, 2009, Fast and accurate short read alignment with Burrows-Wheeler Transform, Bioinformatics 25:1754; and Li, 2011, Improving SNP discovery by base alignment quality, Bioinformatics 27:1157. Assembled sequence reads may be aligned to a reference.

Aligned or assembled sequence reads may be analyzed for the presence of variants, e.g., mutations described, or “called” as variants of a given reference. Mutation calling is described in U.S. Pub. 2013/0268474. In certain embodiments, analyzing the reads includes assembling the sequence reads and then genotyping the assembled reads. In certain embodiments, reads are aligned to hg18 on a per-sample basis using Burrows-Wheeler Aligner version 0.5.7 for short alignments, and genotype calls are made using Genome Analysis Toolkit. See McKenna et al., 2010, The Genome Analysis Toolkit: a MapReduce framework for analyzing next-generation DNA sequencing data, Genome Res 20(9):1297-1303 (aka the GATK program).

Mapping sequence reads to a reference, by whatever strategy, may produce output such as a text file or an XML file containing sequence data such as a sequence of the nucleic acid aligned to a sequence of the reference genome. In certain embodiments mapping reads to a reference produces results stored in SAM or BAM file and such results may contain coordinates or a string describing one or more mutations in the subject nucleic acid relative to the reference genome. Alignment strings known in the art include Simple UnGapped Alignment Report (SUGAR), Verbose Useful Labeled Gapped Alignment Report (VULGAR), and Compact Idiosyncratic Gapped Alignment Report (CIGAR). See Ning et al., 2001, SSAHA: A fast search method for large DNA database, Genome Research 11(10):1725-9. These strings are implemented, for example, in the Exonerate sequence alignment software from the European Bioinformatics Institute (Hinxton, UK).

In some embodiments, a sequence alignment is produced—such as, for example, a sequence alignment map (SAM) or binary alignment map (BAM) file—comprising a CIGAR string (the SAM format is described, e.g., in Li, et al., The Sequence Alignment/Map format and SAMtools, Bioinformatics, 2009, 25(16):2078-9). In some embodiments, CIGAR displays or includes gapped alignments one-per-line. CIGAR is a compressed pairwise alignment format reported as a CIGAR string. A CIGAR string is useful for representing long (e.g. genomic) pairwise alignments. A CIGAR string is used in SAM format to represent alignments of reads to a reference genome sequence.

Output from mapping may be stored in a SAM or BAM file, in a variant call format (VCF) file, or other format. In an illustrative embodiment, output is stored in a VCF file. A typical VCF file will include a header section and a data section. The header contains an arbitrary number of meta-information lines, each starting with characters ‘##’, and a TAB delimited field definition line starting with a single ‘#’ character. The field definition line names eight mandatory columns and the body section contains lines of data populating the columns defined by the field definition line. The VCF format is described in Danecek et al., 2011, The variant call format and VCFtools, Bioinformatics 27(15):2156-2158.

The data contained in a VCF file represents the variants, or mutations, that are found in the nucleic acid that was obtained from the sample from the patient and sequenced. In its original sense, mutation refers to a change in genetic information and has come to refer to the present genotype that results from a mutation. As is known in the art, mutations include different types of mutations such as substitutions, insertions or deletions (INDELs), translocations, inversions, chromosomal abnormalities, and others. Variant can be taken to be roughly synonymous to mutation but referring to a genotype being described in comparison or with reference to a reference genotype or genome. For example as used in bioinformatics variant describes a genotype feature in comparison to a reference such as the human genome (e.g., hg18 or hg19 which may be taken as a wild type). Methods described herein may generate data representing one or more mutations, or “variant calls.”

A description of a mutation may be provided according to a systematic nomenclature. For example, a variant can be described by a systematic comparison to a specified reference which is assumed to be unchanging and identified by a unique label such as a name or accession number. For a given gene, coding region, or open reading frame, the A of the ATG start codon is denoted nucleotide +1 and the nucleotide 5′ to +1 is −1 (there is no zero). A lowercase g, c, or m prefix, set off by a period, indicates genomic DNA, cDNA, or mitochondrial DNA, respectively.

A systematic name can be used to describe a number of variant types including, for example, substitutions, deletions, insertions, and variable copy numbers. A substitution name starts with a number followed by a “from to” markup 913. Thus, 199A>G shows that at position 199 of the reference sequence, A is replaced by a G. A deletion is shown by “del” after the number. Thus 223delT shows the deletion of T at nt 223 and 897-999del shows the deletion of three nucleotides (alternatively, this mutation can be denoted as 897-999delTTC). In short tandem repeats, the 3′ nt is arbitrarily assigned; e.g. a TG deletion is designated 1997-1998delTG or 1997-1998del (where 1997 is the first T before C). Insertions are shown by ins after an interval. Thus 200-201insT denotes that T was inserted between nts 200 and 201. Variable short repeats appear as 897(GT)N—N′. Here, 897 is the first nucleotide of the dinucleotide GT, which is repeated N to N′ times in the population. Systematic nomenclature is discussed in den Dunnen & Antonarakis, 2003, Mutation Nomenclature, Curr Prot Hum Genet 7.13.1-7.13.8 as well as in Antonarakis and the Nomenclature Working Group, 1998, Recommendations for a nomenclature system for human gene mutations, Human Mutation 11:1-3. Variant detection can include using a system of the invention.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of evaluating cancer, the method comprising:
providing, to a computing system, a training data set comprising functional biomarker measurements from a plurality of patient samples each having a known cancer status;
associating the functional biomarker measurements with the cancer statuses;
obtaining a sample from a patient suspected of having cancer;
measuring a functional biomarker of one or more live cells isolated from the sample;
inputting data obtained in the measuring step into the computing system;
correlating the data with the cancer statuses; and
reporting results of the correlating step.

2. The method of claim 1, wherein the measuring step includes measuring a mass or mass accumulation or mass accumulation rate for the one or more live cells.

3. The method of claim 2, wherein the mass or mass accumulation, or mass accumulation rate is measured using a device comprising one or more suspended microchannel resonators.

4. The method of claim 1, wherein the sample comprises a fine needle aspirate or biopsy from a patient suspected of having cancer.

5. The method of claim 1, wherein the computing system comprises a machine learning system selected from the group consisting of a random forest, a support vector machine, a Bayesian classifier, and a neural network.

6. The method of claim 1, wherein the computing system comprises an autonomous machine learning system that associates the functional biomarker measurements with the known cancer statuses in an unsupervised manner.

7. The method of claim 6, wherein the autonomous machine learning system comprises a deep learning neural network that includes an input layer, a plurality of hidden layers, and an output layer.

8. The method of claim 6, wherein the autonomous machine learning system represents the training data set using a plurality of features, wherein each feature comprises a feature vector.

9. The method of claim 6, wherein the autonomous machine learning system comprises a random forest.

10. The method of claim 1, further wherein the training data set includes at least one other source of data associated with the known cancer statuses.

11. The method of claim 10, wherein the at least one other data source includes genomic data.

12. The method of claim 1, further comprising operating a machine learning system to learn relationships among cancer statuses, treatment options, depth of response, known treatment efficacies, and progression free survival.

13. The method of claim 12, further comprising selecting, by the machine learning system, one or more recommended treatments for the patient based, at least in part, on the results of the correlating step and learned relationships.

14. The method of claim 12, wherein one or more of the training data set, cancer statuses, treatment options, depth of response, known treatment efficacies, and progression free survival are obtained from one or more publicly available data repositories.
